(19) United States
(12) Patent Application Publication
(10) Pub. No.: US 2025/0037834 A1
(43) Pub. Date: Jan. 30, 2025

(54) Methods and Systems for Dental Treatment Planning

(71) Applicants: Q & M Dental Group Singapore (LTD), Singapore (SG); EM2AI PTE LTD, Singapore (SG)

(72) Inventors: Chin Siau Ng, Singapore (SG); Kai Chuan Chong, Singapore (SG); Yi Leong San, Singapore (SG); Xing Yi Lim, Singapore (SG); Jian Ming Ng, Singapore (SG); Nadia Nathania Santoso, Singapore (SG); Yew Mun Wong, Singapore (SG); Zheng Jian Huang, Singapore (SG)

(21) Appl. No.: 18/716,623

(22) PCT Filed: Dec. 1, 2022
(86) PCT No.: PCT/SG2022/050874

§ 371 Date: Jun. 5, 2024

(30) Foreign Application Priority Data
Dec. 6, 2021 (SG) 10202113528S

(51) Int. Cl. G16H 20/40; A61B 6/51; G06T 7/00; G16H 10/20; G16H 10/60; G16H 50/20

(52) U.S. Cl. CPC G16H 20/40; A61B 6/51; G06T 7/0012; G16H 10/20; G16H 10/60; G16H 50/20; G06T 2207/20084; G06T 2207/30036

(57) ABSTRACT

The present invention relates generally to methods and systems that can be used to analyse the mouth condition of an individual. The methods and systems can be used in the identification of an oral condition or disease to provide a personalised holistic treatment care plan for the individual. More particularly, the present invention relates to methods and systems that employ artificial intelligence capabilities for generating a treatment plan for an identified oral condition or disease.

### FIELD OF THE INVENTION

The present invention relates generally to methods and systems that can be used to analyse the mouth condition of an individual. The methods and systems can be used in the identification of an oral condition or disease to provide a personalised holistic treatment care plan for the individual. More particularly, the field of the present invention relates to methods and systems that employ artificial intelligence capabilities for generating a treatment plan for an identified oral condition or disease.

### BACKGROUND OF INVENTION

Dental radiographs and digital images have been widely used by dentists in finding lesions or monitoring the progress of treatment of an individual's dental condition. With the advantage of digital dental x-ray and images, such as the immediate availability, the lower radiation dose, the possibility of image enhancement, and image reconstruction, etc., the usage of digital dental images has a great increase in the past decade in clinical diagnosis.

However, despite a rapid growing popularity of digital dental images, there is a high probability of miss diagnosis of radiographs even by the experienced dentists. Furthermore, intra- as well as inter-examiner agreement between dentists are very low.

Dental caries, periodontal diseases and missing teeth are some of the most common oral diseases and conditions that will impact the quality of life for individuals and thus pose an important health problem. The effective management of oral conditions or diseases is highly dependent on the decision reasoning which require a great deal of knowledge: risk factors, treatment plan, treatment outcomes, incidence and progression rates etc. All these are open to professional judgment of the health clinicians examining an individual's oral condition. However, the capacity of a health clinicians to always make the correct decision is limited by cognitive functions, like reasoning and memory capacities. These factors result in divergence between decisions made by different clinicians, which is a serious issue because it often results in over-treatment or undertreatment.

There exists a need to limit uncertainty of reasoning and divergence between dental clinicians' decisions through the development of a clinical decision support system (CDSS) for management of oral conditions and diseases such as caries, periodontal disease and missing teeth.

WO201994504 A1 discloses a machine learning system and method to analyse and detect features, i.e. dental pathologies, in dental radiographs. In particular, an object detection is performed based on an image with a real face focused on the mouth area with a bounding box drawn around the mouth area followed by a UNet segmentation model to determine for every pixel if it is a tooth a not. This disclosure is not based on the use of X-ray images to detect both the tooth numbers and their locality concurrently.

CN109528323A discloses an orthodontic method and device based on artificial intelligence. In particular, a Generative Adversarial Network (GAN) model is used to help with the generation of 3D-reconstruction of an individual's teeth set. The GAN model assists to demonstrate the effects from orthodontic problems. CT scans are used to create temporal slices of the mouth unlike X-Ray which has no temporal component.

CN105260598A discloses a dental diagnosis and treatment decision support system and a decision-making method to obtain the most similar recommended cases in the diagnosis of diseases and automatically provide decision data. In particular, a three-level screening method is adopted to obtain the most similar cases in the diagnosis for fast screening speed. The decision support system leverages on case studies where they convert it into numerical representation and determine the match based off Euclidean distance to the case, which is in contrast to being based on medical directives that are rule-based and more deterministic in nature.

In these respects, there is a need to provide an automated system and method that employs artificial intelligence capabilities for dental charting, analysing the oral condition and generating a holistic treatment plan, if necessary.

### BRIEF SUMMARY OF THE INVENTION

A primary object of the present invention is to provide a computer-implemented method for generating a dental treatment plan of a patient that can comprise the steps of: analysing patient data comprising a dental image of the patient using an AI model to generate AI predictions on tooth detection, numbering and dental issues of the patient; populating a dental chart based on the AI predictions and input received from one or more users; generating a completed dental questionnaire using a clinical decision support system (CDSS) based upon the populated dental chart and input received from the one or more users; generating a final treatment plan based on the completed dental questionnaire; and displaying the final treatment plan to the one or more users.

Another object is to provide a computer system comprising a processor configured to perform the method disclosed herein.

An additional object is to provide a computer-readable storage medium comprising instructions which, when the program is executed by a computer, cause the computer to carry out the method disclosed herein.

Another object is to provide a system for generating a dental treatment plan of a patient that can comprise: an input unit for receiving a dental image; a computer-readable storage medium configured to store instructions defining an AI model; a first server to execute a clinical decision support system (CDSS); a second server to execute the instructions defining the AI model, wherein the second server is configured to perform operations comprising: generate AI predictions on tooth detection, numbering and dental issues of the patient, based upon the dental image using the AI model; and populate a dental chart based on the AI predictions and input received from one or more users, wherein the first server is configured to perform operations comprising: generate a completed dental questionnaire based upon the dental chart and input received from one or more users using the CDSS; and generate a final treatment plan based on the completed dental questionnaire using the CDSS; and an output unit configured to communicate the dental chart, questionnaire and final treatment plan to the user.

Further objects of the invention will appear as the description proceeds.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### Definitions

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

The following words and terms used herein shall have the meaning indicated:

The term “mouth condition” or “mouth diseases” can include but are not limited to caries, periodontal problem, missing tooth, crown, bridge, implant, filling, onlay, inlay, veneer, failed or defective restoration (filling, crown, bridge, veneer, inlay, onlay), root canal treatment, post and core, impacted tooth, partially erupted tooth, unerupted tooth, primary tooth, fracture, periapical lesion, retained root, oral and maxillofacial pathology, oral anatomical landmark, an arrangement of a patient's teeth that is undesirable according to applicable orthodontic standards and/or combinations thereof. An arrangement of teeth can be undesirable for medical, orthodontic, aesthetic, and other reasons, such as overbites, crossbites, openbites, overjets, underbites, and the like.

The term “application programming interface” or “API” refers to a set of subroutine definitions, protocols and tools for building software. In general terms, it is a set of clearly defined methods of communication between various components.

The term “cloud” or “cloud computing” refers to an information technology (IT) paradigm that enables ubiquitous access to shared pools of configurable system resources and higher-level services that can be rapidly provisioned with minimal management effort, often over the Internet.

The term “convolutional neural network” (“CNN”) is as conventionally used in the technical field and generally refers to powerful tools for computer vision tasks, whereby deep learning CNNs can be formulated to automatically learn mid-level and high-level abstractions obtained from raw data such as images. A convolution layer can contain one or more convolution kernels, which each have an input tensor, which can be the same, but which have different coefficients corresponding to different filters. Each convolution kernel in a layer produces a different output map such that the output neurons are different for each kernel. The convolutional networks can also include local or global “pooling” layers which combine the neuron group outputs of one or more output maps. The combination of the outputs can consist, for example, in taking the maximum or average value of the outputs of the group of neurons, for the corresponding output, on the output map of the “pooling” layer. The “pooling” layers make it possible to reduce the size of the output maps from one layer to the other in the network, while improving the performance levels thereof by making it more tolerant to small deformations or translations in the input data. An exemplary pseudocode algorithm is shown below of how a basic CNN works.

Algorithm 1: Convolution Pseudocode

```
Input : Image of [symbol] (x, y) of width W and height H
        Kernel of k(x, y) with dimensions of (2W [symbol]  + 1, 2H [symbol]  + 1)
        with the center of the kernel being coordinate (0, 0)
Output: New image of g(x, y)
procedure Convolution
  for y = 0 to H do
  | for x = 0 to W do
  | | accumulator = 0;
  | | for j=H [symbol]  to H [symbol]  do
  | | | for [symbol] =W [symbol]  to W do
  | | | | accumulator = no [symbol]  + k(i, j) * ( [symbol] x [symbol] i, y [symbol] j);
  | | | end
  | | end
  | | [symbol]  (x, y) = accumulator;
  | end
  end [symbol]
indicates data missing or illegible when filed
```

The term “machine learning” (“ML”) refers to an application of artificial intelligence (AI) that provides systems the ability to learn and improve from experience without being explicitly programmed. Machine learning focuses on the development of computer programs that can access data and use it learn for themselves.

The term “deep learning” (“DL”) is a subfield of machine learning concerned with algorithms inspired by the structure and function of the brain called artificial neural networks. The often cited benefit of deep learning models is their ability to perform automatic feature extraction from raw data, also called feature learning.

The term “module” refers to a self-contained unit, such as an assembly of electronic components and associated wiring or a segment of computer software, which itself performs a defined task and can be linked with other such units to form a larger system.

The term “server” includes any suitable hardware and/or software system, mechanism or component that processes data, signals, or other information. The server may include a general-purpose central processing unit (CPU), a multi-processing unit, a dedicated circuit that implements a specific function, or other system. The process need not be limited to geographic locations or have time limits. For example, the server can perform functions in “real time”, “offline”, “batch mode”, and the like. Some of the processing can be performed at different times and places by another (or the same) processing system. Examples of server systems can include clients, end-user devices, routers, switches, network storage, and the like. The computer can be any server that communicates with memory. The memory is any suitable processor readable storage medium, such as random-access memory (RAM), read-only memory (ROM), hard-disk drive (HDD) or solid-state disk (SSD), or other tangible medium, suitable for storing instructions to be executed by the server.

Unless specified otherwise, the terms “comprising” and “comprise”, and grammatical variants thereof, are intended to represent “open” or “inclusive” language such that they include recited elements but also permit inclusion of additional, unrecited elements.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a diagram illustrating an embodiment of the various computational hardware of the systems and methods disclosed herein.

FIG. 2 is a diagram illustrating an embodiment of the structure of modules used in the second server disclosed herein and their operational relationship with each other and the hardware of the computer system.

FIG. 3 is a representative workflow of the general steps and processes encompassed by the methods disclosed herein for generation of a treatment plan for a patient.

FIG. 4 is a representative flowchart of the system and operational relationship between computational hardware, software components as well as the input from the user regarding the dental chart, dental questionnaire and treatment plan options.

FIG. 5 is a diagram illustrating an embodiment of the structure of modules used in training the deep learning CNNs disclosed herein and their operational relationship with each other and the hardware of the computer system.

FIG. 6A is a diagram illustrating an embodiment of the CDSS and first server disclosed herein and their operational relationship with each other; and FIG. 6B is a representative flowchart illustrating the operational relationship of the CDSS between computational hardware, software components as well as the input from the user and how they interact with the CDSS.

FIG. 7A-F are representative screenshots of a dashboard provided to users via the output unit that show an exemplified presentation of the questionnaire and final treatment plan.

FIG. 8 is an example of an X-Ray image input into the method and system disclosed herein.

FIG. 9 is an example of a dental chart that is populated in the method and system disclosed herein.

### DETAILED DESCRIPTION OF THE INVENTION

In a following description, reference is made to the accompanying drawings which form a part hereof, and in which is shown by way of illustration a specific example in which the invention may be practiced. It is to be understood that other embodiments may be utilized, and structural changes may be made without departing from the scope of the present invention.

Disclosed herein are computer-implemented methods and systems for analysing the condition of a patient's mouth to assist in the diagnosis and provision of a treatment plan for said patient. The methods and systems disclosed herein provide the patient with knowledge of the condition of their mouth in a visually simple manner to reduce the risk of “undetected” dental problems so that the patient will receive treatment as early as possible. As will be appreciated, the early detection and treatment of dental problems and mouth conditions or diseases can prevent costly treatment in future as a consequence of late detection. Thus, the diagnosis and treatment plan generated will not be solely based a dental professional's (e.g. dentist) analysis that can greatly vary due to the strength of that professional's training, capability and educational background.

The methods and systems disclosed herein aims to provide an end-to-end solution from diagnostic and dental charting up to devising a patient's treatment care by providing insight into the condition of a patient's mouth using artificial intelligence (AI) capabilities that have been developed and trained. The artificial intelligence capabilities employed can analyse a patient's dental image to provide insight and extrapolate clinically pertinent information that may have been missed out by manual effort of dental professionals.

To generate an ethical treatment plan options for a patient, artificial intelligence (AI) can be employed in combination with a CDSS. In this regard, the mouth condition of a patient can be analysed by an AI model to identify mouth conditions or diseases with the output being fed into a CDSS for assisting in a diagnosis and generation of personalised treatment plan options.

It will be appreciated that the performance of software-only systems in the analysis of patient data (i.e. dental images) often falls short of that which is needed for accurate results and prevention of false readings. Thus, in addition to employing artificial intelligence capabilities, the methods and systems disclosed herein can utilise the knowledge and input of dental professionals. In particular, to facilitate the decision-making process and generation of a holistic treatment plan, the methods and systems disclosed herein can utilise input from dental professionals (i.e. the user) in performing certain updates, decisions, and selections that the software components alone would otherwise be unable to perform or would perform poorly. In this regard, the methods and systems disclosed herein represent a symbiotic human-machine approach in the analysis of a patient's mouth condition that combines the respective strengths of computer and human processing of data, while minimizing the amount of human involvement required. Consequently, the resulting diagnosis and treatment plan for the patient can be generated on the combined basis of artificial and human intelligence.

As will be appreciated, the system and method described herein can be implemented on a computer using a combination of both hardware and software. Various aspects can be implemented on programmable computers, each computer including a one or more input unit, a data storage medium, a hardware processor and an output unit or communication interface. It should be appreciated that the use of terms such as servers, services, units, modules, interfaces, portals, platforms, or other systems formed from computing devices is deemed to represent one or more computing devices having at least one processor configured to execute software instructions stored on a computer readable storage medium. For example, a server can include one or more computers operating as a web server, database server, or other type of computer server in a manner to fulfil described roles, responsibilities, or functions.

In one embodiment, the system and method described herein can include one or more input units for input of at least one dental images, one or more computer-readable storage mediums configured to store instructions defining an AI model and a CDSS; one or more servers to execute the instructions defining the AI model and the CDSS; one or more databases; one or more cloud storage systems; and one or more output units.

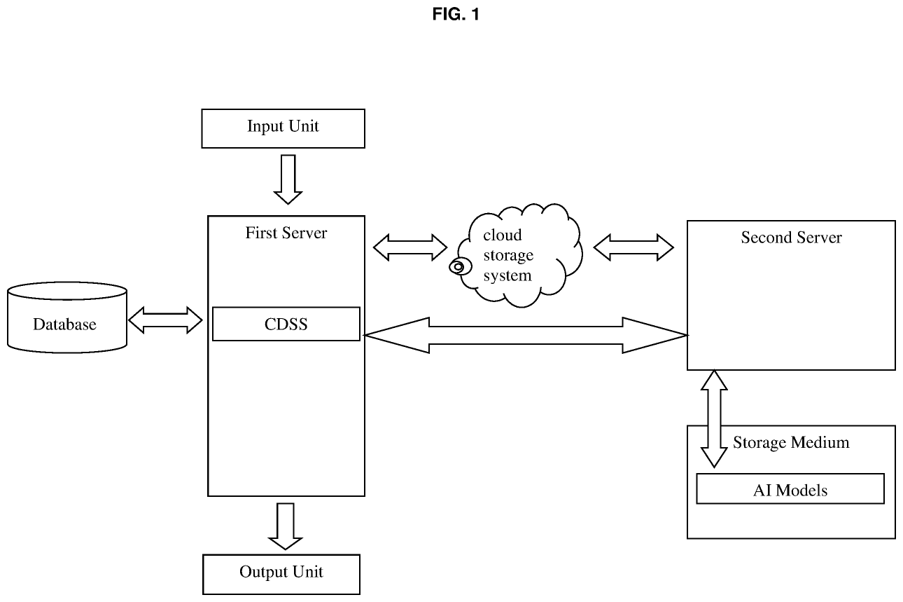

FIG. 1 illustrates a representative embodiment of the computational hardware that can be employed in the methods and systems disclosed herein. In one embodiment, the methods and systems disclosed herein can generally include an input unit for receiving and inputting a dental image from a user to a first server. A second server can be in communication with the first server to access and execute AI models stored as software instructions in one or more computer-readable storage medium. The AI models can be in communication with both a database and cloud storage system via the first and second servers. The first server can also comprise and execute a CDSS. An output unit can be included and configured to communicate with the user via the first server.

In one embodiment, the user can include, one or more individuals, one or more patients, one or more dental clinicians or clinical professional, one or more physicians and any other stakeholder/concerned individual.

In one embodiment, the input unit of the system can include any computational device or input module capable of receiving input from one or more databases, cloud storage systems or image capturing device. In one embodiment, the dental image can be input by a user through the input unit or by integration of an image capturing device with the computer system. For example, the computer system can be integrated with an X-ray machine to read a radiography into the computer system. The input dental image can then be displayed via an output unit (i.e. user interface) for review.

In one embodiment, the first server can be a web-based database server, such as an Integrated Dental Management System (IDMS) server.

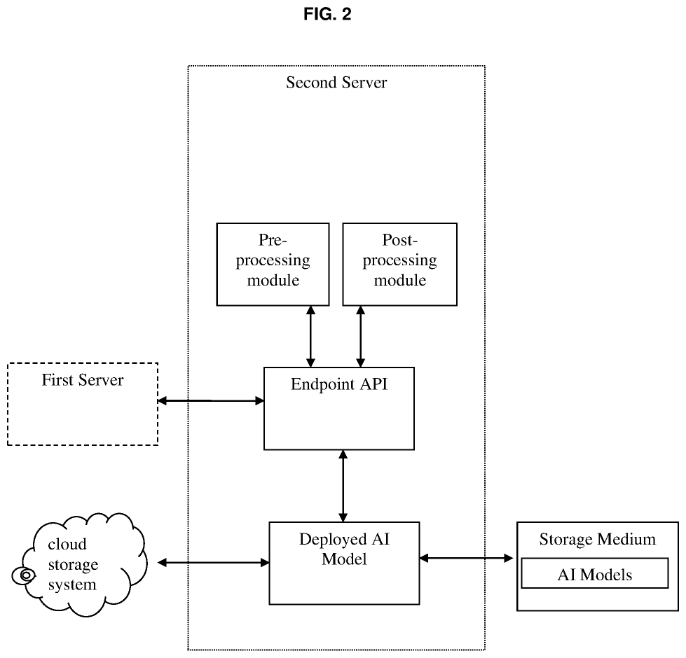

In one embodiment, the second server can be an AI server. The AI Server can be any conventionally used Virtual Machine that has a CPU power capable of executing the AI model disclosed herein for inference of the input dental image and to provide the AI predictions for populating a dental chart. FIG. 2 illustrates a representative embodiment of the structure of the second server to deploy the AI model with various communication links for operational relationship between software and hardware components of the computer system. In particular, the second server can have access or communicate with the first server, the database via the first server and the cloud storage system for analysing the dental image using the AI model. In one embodiment, the second server can include a pre-processing module, post-processing module, an application programming interface (API) endpoint module for deploying or executing the AI model to provide the AI predictions. The pre-processing module can convert the dental image into a suitable format to be used by the AI model. The post-processing module can convert the raw output from the AI model into a suitable format used by the first server along with determining where all the AI predictions belong with respect to the teeth.

In one embodiment, the database can function as a local memory store, which can be a memory cache with persistence. The database can also be a disk storage based database: relational, noSQL and/or others. Data input from and output into the database can be routed by one or more internal API's. The data stored in the database can include, but is not limited to, the AI predictions output by the AI models, a model registry of the AI models that have been trained, a model deployment log of the AI models deployed, and an audit log of the AI predictions for individual dental images. As elaborated on below, the AI predictions can be in the form of bounding box data.

In one embodiment, the cloud storage system can store data from the first and second servers through links that can be based on a communication spectrum such as WiFi, Bluetooth, Sigfox, Lora, IoT, cellular, and/or others. One or more APIs can allow data transfer between the cloud storage system and the first and/or second servers. The one or more APIs can communicate with the cloud storage system for routing data and requests. In one embodiment, the data stored on the cloud storage system can include but is not limited to, the raw dental images, TF (TensorFlow) record files, AI model weights, log files for the AI model for tracking and patient data. In this regard, the TF record files refer to the dental images processed through the AI model with bounding box data indicated and optimised for consumption by a Tensorflow framework. The patient data can include data on the medical history, dental history, social history of the patient and biodata (e.g. age, gender). The AI model weights can refer to the derived mathematical values in the mathematical functions of the AI model following training of the AI models.

In one embodiment, the output unit can communicate or display on a user terminal the output of one or more stages of the method disclosed herein in addition to the final treatment plan result. In this regard, the output can be displayed via the first server, more specifically the IDMS server, whereby one or more APIs communicate with the first server on demand to display the output via the output unit. In one embodiment, the output unit can communicate or display a dashboard to the user representing the output of one or more stages of the method disclosed herein in addition to the final treatment plan result.

In one embodiment, the output unit can include a graphical user interface (GUI). In one embodiment, the GUI can have the facility to load and display the patient data, a dental chart, a dental questionnaire, treatment plan options and a final treatment plan. In one embodiment, the output unit can allow the user to selectively configure or update the output from the system relating to the patient data, dental chart, a dental questionnaire and treatment plan options. In one embodiment, the GUI can include a dashboard display system that allows a user to selectively configure or update the output from the system. The dashboard can include an interactive database of menu items that is accessible by the user for selecting the items to be displayed using touch screen technology or actuatable buttons to select the desired displayed items and the appearance of the displayed items.

The architecture design of the computer system disclosed herein provides APIs for creating, accessing and consuming data derived from the user and processing of the dental image via the AI models and CDSS, as well as the training of the AI models. In one embodiment, APIs utilised in the system disclosed herein can include an endpoint API, Object Detection API, inference API, a training API and a CDSS API. As will be appreciated, one or more additional APIs can link the database and cloud storage system to transfer data to and from the servers.

Further, the computer system disclosed herein can include additional components. For example, the system can include one or more communication channels or interconnection mechanism such as a bus, controller, or network, that interconnects the components of the system. In various embodiments of the operating system software provides an operating environment for various software's executing in the computer system and manages different functionalities of the components of the computer system. The communication channel(s) allow communication over a communication medium to various other computing entities. The communication medium provides information such as program instructions, or other data in a communication media. The communication media can include wired or wireless methodologies implemented with an electrical, optical, radiofrequency, infrared, acoustic, microwave, Bluetooth® or other transmission media.

In one embodiment, there is provided a system for generating a dental treatment plan of a patient that can comprise: one or more input units for receiving a dental image; one or more computer-readable storage mediums configured to store instructions defining an AI model and a CDSS; one or more servers to execute the instructions defining at least one AI model and the CDSS; a database; a cloud storage system; and an output unit configured to communicate the results to the user, wherein the one or more servers can be configured to perform operations comprising: generate AI predictions based upon the dental image using the AI model; populate a dental chart based on the AI predictions and input received from one or more users; generate a completed dental questionnaire using a CDSS based upon the dental chart and input received from one or more users using the CDSS; and generate a final treatment plan based the completed dental questionnaire using the CDSS.

In another embodiment there is provided a system for generating a dental treatment plan of a patient that can comprise: an input unit for receiving a dental image; a computer-readable storage medium configured to store instructions defining an AI model and a CDSS; a first server to populate a dental chart based on AI predictions and user input, and to execute the instructions defining the CDSS for generating a completed dental questionnaire and a final treatment plan based on user input; a second server to execute the instructions defining the AI model to generate the AI predictions; a database; a cloud storage system; and an output unit configured to communicate the results to the user.

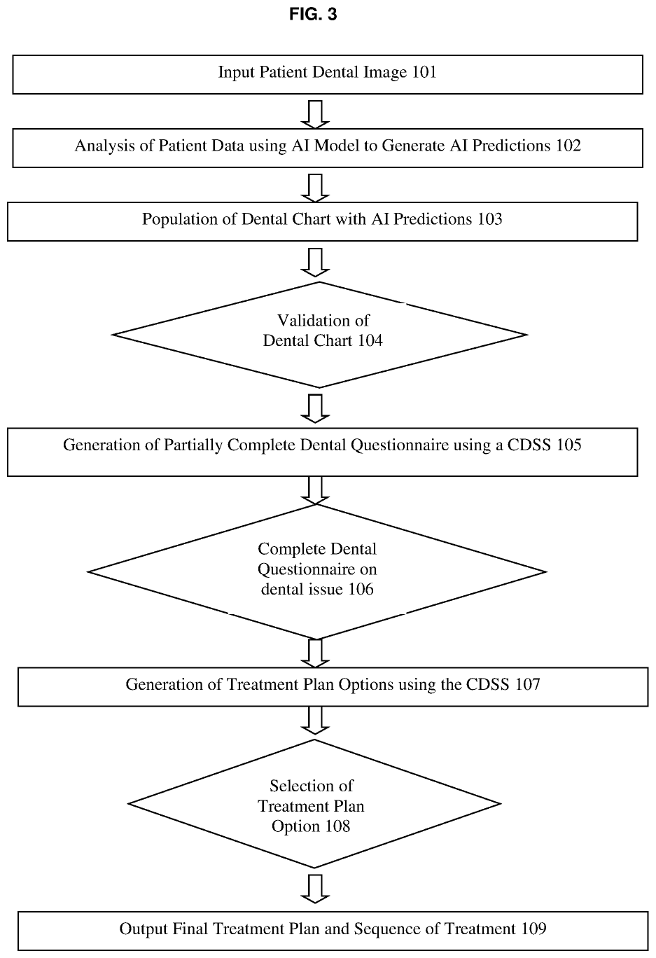

FIG. 3 illustrates a representative workflow and steps involved in the method that can be implemented on the computer system disclosed herein for generation of a treatment plan for a patient.

The analysis of a patient's mouth condition and generation of a treatment plan begins at step 101 with the input of patient dental image into a first server to initially process. The input patient dental image can be communicated to a user by the first server via an output unit, such as a graphical user interface.

The patient's dental image can be a radiograph such as an X-ray image. In one embodiment, the X-ray image can be a bitewing image, periapical image or an orthopantogram/panoramic image.

At step 102, at the request of the user, the first server can instruct a second server to execute AI model to process the dental image and generate AI predictions. The AI predictions can be based on the combined output or inference results of the AI model to provide predictions on the tooth numbering, dental issues and restorations. For example, the AI predictions can be based upon the following components: 1) bounding box x-min, y-min coordinates as well as the width and height (x-min and y-min is the top-left corner coordinates of the bounding box); 2) the probability of an object being present in the bounding box; and 3) the class probabilities of the object detected in the bounding box. An exemplary pseudocode of this process before sending it to step 103, is shown below.

Algorithan 2: Inference Pipeline Pseudocode

```
Input : I where I = Input Image
Output : J where J = JSON of teeth with conditions/restorations
          assigned to them
Required: M, where M = {m₁, m₂,...,mₙ} where mᵢ is a deep learning
        object detection model used in the system
procedure InferencePipeline;
Initialize B, where B is an empty array to store the model results that
contain the bounding box coordinates, classification class and the
clasification score;
Initialize R, where R is an empty array to store the final assigned
bounding [symbol]  to tooth results.
Initialize C = {Tooth decay, Filling}, where C conains the classes that
requires surface location on tooth to be specified.;
I_{processed} = preprocessingFunction(I);
for mᵢ ϵ M do
| modelResults ← mᵢ(I_{processed})
| filteredModelResults ← removeBoxesBelowThreshold(modelResults);
| Push filtersModeResults into B;
end
toothBoundingBoxes ← extractToothBoundingBoxes(B);
for b ϵ B\{toothBoundingBoxes} do
| currentBoxIOUResults ← getHighestIOUWithToothBoundingBoxes
| (b, toothBoundingBoxes);
| if class in C for current [symbol]  then
| | currentBoxIOUResults ←
| | assignSurfaceBasedOnPositionOnAssignedTooth
(currentBoxIOUResults)
| end
| Push currentBoxIOUResults into R;
end
J ← convertToJSON(R) return J [symbol]
indicates data missing or illegible when filed
```

At step 103, the AI predictions are transmitted from the second server to the first server. The first server populates a dental chart based upon the received AI predictions. At step 104, the populated dental chart can be communicated to the user via a user interface for the purpose of checking the dental chart and information presented therein. If the dental chart needs modifying or updating with new clinical information, patient data, examination results or to correct discrepancies, the user can selectively edit the dental chart. The final updated dental chart can be confirmed and validated by the user for further processing by the first server.

The user interface can include elements for the user to confirm or reject the generated AI predictions, or add information for any tooth.

The user interface displaying the dental chart can indicate teeth detected with numbering in addition to any dental issues indicated by visual elements and/or labels on the dental chart. The locations and dimensions of the visual elements can be visually distinguished according to the type of dental issue detected. For example, non-pathological conditions may be marked on the image in a first colour, while pathological conditions are marked using a different colour. The user interface can include user interface elements to permit the user to add or remove visual elements based upon their physical review and assessment of the patient's mouth.

A standard symbolic numbered dental chart can be displayed, comprising a plurality of teeth representing the typical arrangement of a full complement of adult teeth or primary teeth as appropriate, mapped or correlated to tooth position, number, and detected dental issues. The dental chart can be colour coded to signal the location of the dental issue (e.g. surface or tooth level). In addition, teeth that appear in the standard chart that are not detected (e.g. missing tooth) can also be shown as blacked-out. Thus, teeth depicted symbolically in the dental chart can be correlated to the regions of interest identified by the bounding boxes or other visual elements. Teeth that are detected as but otherwise exhibit no dental issues (i.e., “normal” teeth) can be omitted from the dental chart, or included and indicated as being normal.

In one embodiment, the dental chart can be accompanied by a table listing the AI predictions for each tooth.

A final dental chart can be generated after the user has reviewed and amended, when needed, the populated dental chart and information therein.

At step 105, based upon the final validated dental chart, the first server instructs and executes the CDSS to generate a partially complete dental questionnaire. In this context, “partially” can refer to an incomplete questionnaire whereby one or more questions remain unanswered. The questionnaire can be partially complete due to some of the answers being automatically extracted from both patient data stored in the cloud storage system and the final dental chart.

The first server can communicate the partially complete dental questionnaire to the user containing both answered and unanswered questions. At step 106, the user can check the answered questions and complete the unanswered questions to provide a completed dental questionnaire for the first server to further process.

In one embodiment, the questions can be in the format of single and/or multiple-choice questions related to the location of the dental issue, the symptoms of the dental issue, patient biodata and treatment options. In one embodiment, the questions can be in the form of ‘yes’ or ‘no’ questions in addition to single and/or multiple choice questions.

In one embodiment, the questionnaire can be presented to the user via the user interface or dashboard, with separate interactive sections (tabs) for patient biodata, each dental issue indicated in the dental chart and treatment options. In one embodiment, the questionnaire can include a section for general patient biodata and a section for each dental issue. Each of these sections can include a pre-set series of questions dependent on the dental issue for the user to answer. The section for general patient biodata can include questions on the patient's medical history, dental history, social history, general dental risk factors and current symptoms being presented by the patient (e.g. fever, bad breath, bleeding gums, generalised pain etc). The section for each dental issue can include a representation of the populated dental chart indicating the tooth number applicable to said dental issue, the location of the dental issue at the surface level or tooth level (e.g. mesial, occlusal, distal, buccal, lingual) and one or more questions related to the symptoms of the dental issue.

In one embodiment, the questions related to the symptoms of the dental issue can be presented one at a time in a series, whereby question number 2 will only appear once the answer to question 1 has been selected. Accordingly, the CDSS can be in a rule-based framework with the questions being generated in the form of a directed graph.

At step 107, the first server can instruct the CDSS to process the completed dental questionnaire and generate a preliminary diagnostic assessment on the patient's mouth condition with one or more treatment plan options.

The diagnostic assessment and one or more treatment plan options are communicated to the user for review via the output unit (i.e. dashboard). In particular, for each diagnostic assessment identified, the CDSS can propose one or more treatment plans, corrective appliances, or combinations thereof. The diagnostic assessment and treatment plan can be generated separately for each tooth identified with an issue and the mouth collectively.

At step 108, a treatment plan option is selected by the user for each tooth identified with a dental issue. The selection of the treatment plan option can be at the discretion of the user or following consultation between the user and the patient in deciding the most suitable treatment plan. In this regard, the user can consider patient preferences and/or dentist-specified preferences in selecting the treatment plan options. For example, a patient can filter the proposed treatments and corrective appliance results based on cost, risk vs benefit, side effects, personal preference, pain, difficulty in eating or the relative aesthetics of treatment. Similarly, a dentist can filter the proposed treatments based on ideally in consideration of the overall patient's condition including oral condition, medical condition, socioeconomic condition, dietary and behaviour, environmental condition, risk and prognosis of the treatment options.

At step 109, the treatment plan option for each identified dental issue selected by the user is input to the first server. The first server instructs and executes the CDSS to process the selected treatment plan options and generate a final treatment plan along with the sequence of steps for said treatment. The final treatment plan will be communicated to the user via the output unit.

The sequence of treatment steps can be in the form of one or more phases including but are not limited to an “urgent phase”, “control phase”, re-evaluation phase”, “definitive phase” and a “maintenance phase”. The “urgent phase” relates to the need for an emergency treatment of an identified dental issue, however, as will be appreciated the treatment plan may not include an “urgent phase” if no emergency treatment is required.

Accordingly, in one embodiment there is provided a computer-implemented method for generating a dental treatment plan of a patient that can comprising the steps of: analysing a dental image of the patient using an AI model to generate AI predictions; populating a dental chart based on the AI predictions and input received from one or more users; generating a completed dental questionnaire using a CDSS based upon the populated dental chart and input received from the one or more users; generating a final treatment plan based on the complete dental questionnaire; and displaying the final treatment plan to the one or more users.

In another embodiment, there is provided a computer-implemented method for generating a dental treatment plan of a patient that can include the steps of: inputting a dental image; transmitting the dental image from the first server to a second server and instructing the second server to execute an AI model to generate AI predictions; instructing the first server to populate a dental chart based on the AI predictions and input from the user; instructing the first server to execute a CDSS and generate a completed dental questionnaire based upon the validated dental chart and input from the user; instructing the first server to execute the CDSS to generate a final treatment plan based on the complete dental questionnaire; and displaying the final treatment plan to the user.

In one embodiment, there is provided a computer program comprising instructions which, when the program is executed by a computer, cause the computer to carry out the method disclosed herein.

In one embodiment, there is provided a computer system comprising a processor configured to perform the method disclosed herein.

In one embodiment, there is provided a computer-readable storage medium comprising instructions which, when the program is executed by a computer, cause the computer to carry out the method disclosed herein.

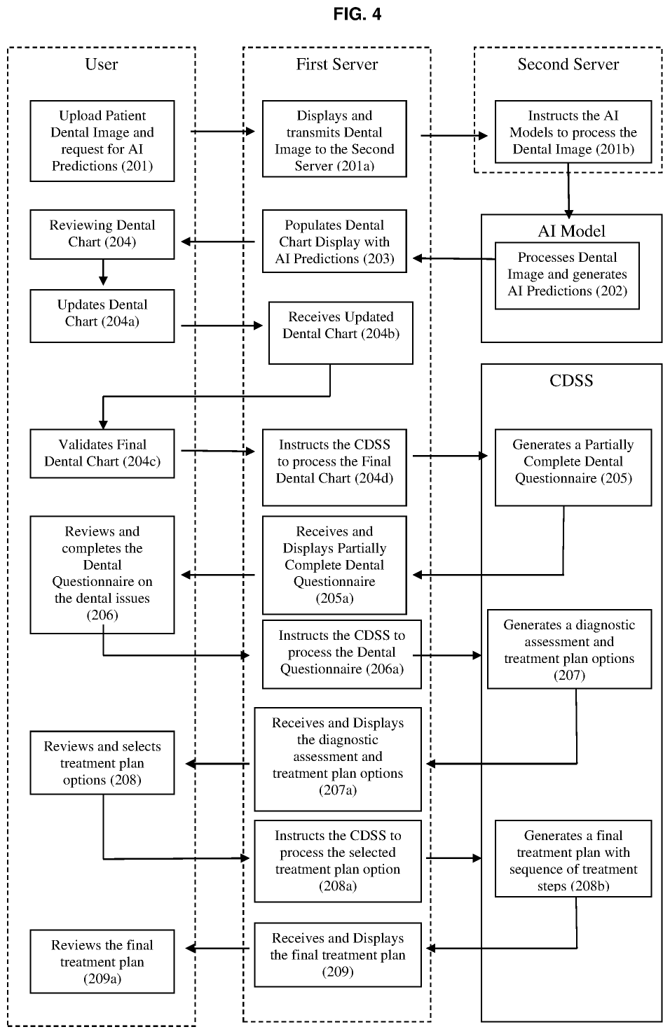

FIG. 4 is a representative flowchart of the system and operational relationship between computational hardware, software components as well as the input from the user with regard to populating and validating the dental chart, generating the completed dental questionnaire and the treatment plan options followed by the final treatment plan. In particular, FIG. 4 further defines the method disclosed in FIG. 3 by the inclusion of intermediary steps.

At step 201, the patient dental image is input into a first server to be initially processed. At step 201a, the input patient dental image can be communicated to a user by the first server via an output unit and also transmitted to the second server. At step 201b, at the request of the user, the first server instructs the second server to process the dental image through the AI model.

At step 202, the AI model processes the dental image and generates AI predictions as an output.

At step 203, the AI predictions are transmitted from the second server to the first server. The first server populates a dental chart based upon the received AI predictions.

At step 204, the populated dental chart can be communicated to the user via a user interface for the purpose of reviewing and checking the dental chart and information presented therein. At step 204a, if the dental chart needs modifying or updating with new clinical information, patient data, examination results or to correct discrepancies, the user can selectively edit the dental chart. At step 204b, the updated and final dental chart can be received and further processed by the first server. At step 204c, the updated dental chart can be communicated to the user via a user interface for the purpose of further reviewing and checking. If the updated dental chart needs no further modification or updating, the user can confirm and validate the final dental chart for further processing by the first server. At step 204d, at the request of the user, the first server can instruct and execute the CDSS to process the final dental chart.

At step 205, the CDSS generates a partially complete dental questionnaire that is transmitted to the first server. At step 205a, the partially complete dental questionnaire can be received by the first server and communicated to the user via a user interface for the purpose of reviewing and checking.

At step 206, the user can check the answered questions and complete the unanswered questions to submit a completed dental questionnaire for the first server to further process. At step 206a, at the request of the user, the first server can instruct the CDSS to further process the completed dental questionnaire.

At step 207, the CDSS processes the completed dental questionnaire and generates a preliminary diagnostic assessment on the patient's mouth condition with one or more treatment plan options. At step 207a, the preliminary diagnostic assessment and treatment plan options can be received by the first server and communicated to the user via the user interface for the purpose of reviewing and checking.

At step 208, the user reviews the preliminary diagnostic assessment and selects a treatment plan option(s). At step 208a, at the request of the user, the first server can instruct the CDSS to further process the selected treatment plan option(s). At step 208b, the CDSS processes the selected treatment plan option(s) and generates a final treatment plan along with the sequence of steps for said treatment.

At step 209, the final treatment plan along with the sequence of steps for said treatment can be received by the first server and communicated to the user via a user interface. At step 209a, the user can review the final treatment plan along with the sequence of steps.

### Artificial Intelligence (AI) Model

Artificial Intelligence imaging recognition can be used to analyse a patient's dental image, such as an Orthopantomogram image, to determine the condition of the mouth. The artificial intelligence model disclosed herein can combine image recognition and localisation for object detection and incorporate the analysis results into an automatic tooth charting system. The analysis results and output of the AI model disclosed herein can provide AI predictions. The AI predictions can be based upon satisfying a confidence threshold.

To generate the AI predictions, the AI model can automatically perform object detection of an input dental image to detect and identify teeth locality for numbering and classification of the teeth relating to dental issues. In one embodiment, the AI model can additionally perform keypoint detection. The locality of teeth and tooth numbering can be determined by forming of bounding boxes around teeth. The classification of the teeth can be based upon object detection in the bounding boxes. In object detection, the AI model primarily focus on extracting features to help detecting the bounding box area along with a likelihood that there is an object present in that said area and the likelihood of the object relating to a dental issue itself.

The AI predictions can be based upon the following components: 1) bounding box x-min, y-min coordinates as well as the width and height (x-min and y-min is the top-left corner coordinates of the bounding box); 2) the probability of an object being present in the bounding box; and 3) the class probabilities of the object detected in the bounding box.

In this regard, the bounding boxes can define a boundary for each tooth captured in the dental image. Subsequently, a number can be assigned to each detected tooth. Numbering can be assigned in accordance with the FDI notation, the Universal Numbering System, the Palmer notation method, or any other suitable dental notation. It will be appreciated that the tooth numbering can be based on an adult dental chart, although a primary dental chart and appropriate notation can be employed for younger patients.

The bounding boxes formed around teeth can indicate different arrangements of teeth in the dental image, for example a first bounding box around anterior teeth, a second bounding box around left posterior teeth, a third bounding box around right posterior teeth, and a fourth bounding box around all teeth. In addition, the bounding boxes can include data on the location of the object detected (e.g. dental issue) being at the surface level or tooth level (e.g. mesial, occlusal, distal, buccal, lingual).

Accordingly, the AI predictions as an output of the AI model can be one or more probabilities. In one embodiment, the output of the AI models can act as a classifier and assign a probability for detecting a dental issue on located teeth in the dental image. The threshold and probability value range (i.e. ranging from 0 to 1) for each classification and dental issue can vary and be suitably set.

In one embodiment, the classification can refer to dental issues including but not limited to caries, periodontal problem, missing tooth, crown, bridge, implant, filling, onlay, inlay, veneer, failed or defective restoration (filling, crown, bridge, veneer, inlay, onlay), root canal treatment, post and core, impacted tooth, partially erupted tooth, unerupted tooth, primary tooth, fracture, periapical lesion, retained root, oral and maxillofacial pathology, oral anatomical landmark, an arrangement of a patient's teeth that is undesirable according to applicable orthodontic standards and/or combinations thereof. An arrangement of teeth can be undesirable for medical, orthodontic, aesthetic, and other reasons, such as overbites, crossbites, openbites, overjets, underbites, and the like. In one embodiment, the classification can be caries, periodontal disease region and restorations. Each dental issue can be at different stages and progress and/or severity of development.

Accordingly, the AI model can perform the detection and numbering of teeth as well as detecting and classifying non-pathological and pathological dental issues from the input dental image.

In one embodiment, the AI model can include one or more deep learning CNNs for object detection, whereby each deep learning CNN can be used for detecting a different dental issue. In one embodiment, each deep learning CNN can be used for detecting objects that are dynamic in size (e.g. missing tooth, tooth decay and gum disease, and any tooth restorations or filings) or static in size (e.g. tooth location/numbering and impacted teeth).

In one embodiment, the AI model can include two or more deep learning CNNs for object detection, whereby each deep learning CNN can operate in parallel to separately process the same input dental image, as opposed to sequentially or in an order. In this regard, the dental image can be input into each deep learning CNN for simultaneous processing and providing separate output predictions, that can be combined to form a consolidated AI prediction. For example, a first deep learning CNN can be used for detecting tooth location/numbering, a second deep learning CNN can be used for detecting caries, a third deep learning CNN can be used for detecting restorations, a fourth deep learning CNN can be used for detecting primary tooth, a fifth deep learning CNN can be used for detecting impacted tooth and a N^{th} deep learning CNN can be used for detecting any one of the other dental issues.

In one embodiment, the machine learning CNNs can be deep learning CNNs. In one embodiment, the AI model can include two or more deep learning CNNs. In another embodiment, the AI model can include two deep learning CNNs.

A first deep learning CNN can be used for detecting objects that are dynamic in size. A second deep learning CNN can be used for identifying objects that are static in size. In one embodiment, both the first and second deep learning CNN can be object detection CNN models.

The first deep learning CNN can have an architecture that predicts objects based on 3 points forming a bounding box for each object for localization and classification. The 3 points can be the top left-hand corner, bottom right-hand corner and the centre which then can be used to form a bounding box for each object. The first deep learning CNN can function on the concept of centre pooling and cascade corner pooling. Centre pooling referring to the maximum value in the horizontal and vertical direction of the feature map; and cascade corner pooling referring to the identification of a boundary max value, an internal maximum value and a summation of these 2 values together.

The second deep learning CNN can have an architecture that is different from that of the first deep learning CNN, wherein the first deep learning CNN predicts objects as points as opposed to predicting objects in regions or boxes. The second deep learning CNN can adopt a bi-directional feature pyramid network (BiFPN) unlike other CNN architectures such as Faster-RCNN. In particular, the second deep learning CNN can have an architecture of a sufficient width to allow finer details to be captured (as smaller objects are bigger in larger images) and of a sufficient depth to allow greater semantics to be extracted from the image itself. The size of the input image can be important and thus a heuristic based scaling mechanism can exist within the second deep learning CNN. To accommodate these requirements the second deep learning CNN can have a compound scaling architecture.

In a preferred embodiment, the deep learning CNNs can include EfficientDet and CenterNet. The EfficientDet model can be used for identifying objects that are static in size, whereas the CenterNet model can be used for detecting objects that are dynamic in size.

Although EfficientDet relies on an anchor mechanism to determine a prior area region of interest for detection, the need to configure anchors properly to serve different sizes can be troublesome. In contrast, CenterNet detects objects by points requires less hyper-parameters to tune and allows more dynamism for predictions which will serve well in the event that the model sees object classes which may have variations in size during out of sample predictions. It will be appreciated that CNNs, other than EfficientDet and CenterNet, can be adopted in the AI model disclosed herein that perform the same function and outcome for object detection.

The deep learning CNNs disclosed herein can be trained using a dataset of patient information representing a variety of mouth structures and mouth conditions (healthy or with dental issues). The CNNs can be trained through deep learning techniques, so that the CNN can be applied to the system and method disclosed herein for accurately and sensitively discriminating the locality and class of the teeth as well as objects that can indicate symptomatic features of dental issues. In particular, the deep learning CNNs can be trained using a large dataset derived from a cohort of patients. Machine learning packages or platforms can be used in training the CNNs. The dataset can include orthopantomogram records of patients coupled with clinical professional examination findings that assist in validating the analytical results of the CNNs.

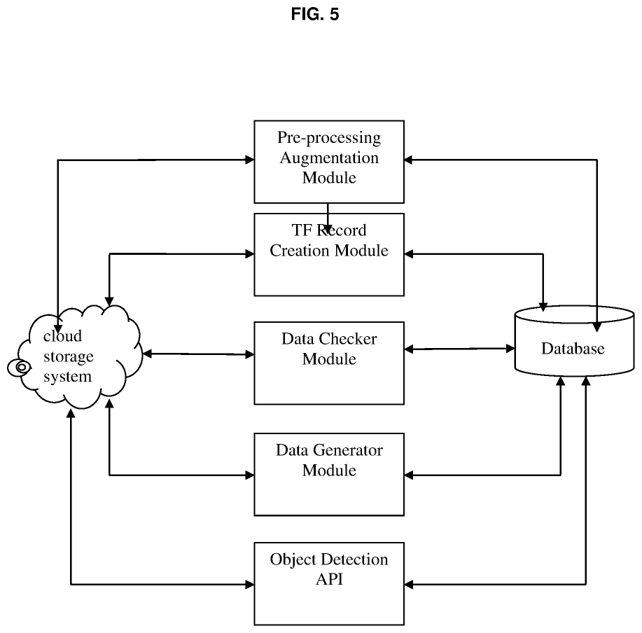

FIG. 5 illustrates a representative embodiment of the structure of modules that can be used in training the deep learning CNNs and their operational relationship with each other and certain hardware of the computer system. In particular, training of the CNNs can include the use of a Pre-processing Augmentation Module, a TF Record Creation Module, a Data Checker Module, a Data Generator Module and a TF Object Detection API.

The pre-processing augmentation module can be used to manipulate dental images for improved object detection, such as cropping unnecessary parts of the image. The TF Record Creation module can be used to convert dental images into a protobuf file (i.e. one file containing all the images) which is optimized within the TensorFlow framework when loading/processing data. The Data checker module can serve to ensure quality labels (e.g. tooth number labels) by performing certain heuristics checks. The Data Generator module can be used to convert XML files labels into a tabular format for easier consumption for the TF Record section and analysis. The Object Detection API can be an open-source library by a search platform, such as Google, where many popular object detection model architectures are coded and can be used.

The modules and API of the training system can each be connected with the database and cloud storage system via a two way link for receiving and transmitting data therebetween. In one embodiment, the modules of the training system can be stored as software instructions in the computer-readable storage medium.

In one embodiment, the deep learning CNNs can be re-trained or optimised periodically as opposed to continually. Accordingly, the deep learning CNNs can be updated and re-trained in a batch process, when required.

Following training of the deep learning CNNs, the output AI prediction results are preferably validated with a cross-validation technique on blinded data sets. In particular, the deep learning CNNs can be processed with a validation set of dental images that were not used for training and are a separate distinct dataset of images to the training dataset. The performance of the deep learning CNNs using the validation dataset can be compared against the training dataset to determine the accuracy of the deep learning CNNs disclosed herein. In this regard, a validation dataset can be used for each CNN used, whereby the outcome can indicate the most suitable CNN to employ in the detection of the dental issues.

The output obtained from the AI models in relation to the detected tooth numbering and dental issues form the AI predictions. These AI predictions can then be transmitted from the second server to the first server to populate a dental chart. In one embodiment, the raw output from the AI models can be in a tensor format which will then be transformed into a json to return to the first server. In particular, the output results from each CNN of the AI model can be combined together along with their probabilities and localities to form the final raw AI model output.

The AI predictions can be used to automatically populate the dental chart in a post-processing step. This automatic population of the dental chart can employ the use of metrics, such as Intersection over Union (IOU). In particular, the IOU refers to the percentage of the area of overlap between 2 bounding boxes with respect to the total area of the union of the 2 boxes as an algorithm to assign the detected classifications (caries, restorations etc.) to the respective tooth for illustration in the dental chart.

### Clinical Decision Support System (CDSS)

Clinical decision support systems (CDSSs) represent a branch of expert systems (ESs) that utilise medical knowledge engineering. The CDSS can use ES design principles to simulate the processes of diagnosis and treatment that are usually done by medical experts. The aim being to assist clinical professionals solve complicated medical problems or make diagnoses.

In a conventional structure of a decision support system, the decision maker or user will arrive at a solution through their interpretation and understanding of the information at hand and the problem. When there is more than one decision maker, the process can be complicated, all the more when the information available can be subjective, objective, a combination of both.

In the specific context of a dental clinic, the problem and solution relates to the dental treatment that best suits the patient. The decision made by the decision maker will depend on the problem itself which would influence the criteria adopted by the decision maker as well as relevant information pertaining to the problem. In most clinical situations, the patient can also act as the decision maker where information in terms of financial cost and aesthetic demands can influence the final outcome of the decision. As dentists are limited by and differ in their cognitive functions, such as in the recall and application of possible risk factors and evaluation, there can be potential differences in the decisions made by different dentists, or even by one dentist at different times. The CDSS disclosed herein can minimize such divergence. Specifically, rather than a subjective analysis or diagnosis based on the individual clinical professionals managing of a patient, the CDSS disclosed herein represents expert knowledge collected across dental expert committee of professors, dental specialists and experienced dental practitioners. Thus, the risk of undertreatment, overtreatment and/or negligence can be reduced with significant benefit to the patient.

Conventionally, a CDSS can be based on a data-driven approach alone, using historical data. While this approach is unbiased and relatively cheaper than a solely knowledge-based approach, the major drawback is that the data driven approach will be limited to a particular kind of treatment plan. To ensure the successful implementation of decision support system, it is important to account for knowledge-based data and information within the domain and technical field.

Accordingly, in one embodiment, the CDSS employed can be a rule-based or knowledge-based CDSS. That is, the CDSS disclosed herein does not employ a data-driven approach and is solely a knowledge-based CDSS to generate the dental questionnaire, diagnosis and treatment planning options.

The CDSS employed into the method and system disclosed herein can advantageously provide an automated diagnosis and treatment planning options system, which allows a user to receive an objective and reliable treatment planning options based on the patient's needs. In this regard, the CDSS disclosed herein can be configured and designed to provide an expert system for the decision-making process by using the specific characteristics of each patient. In one embodiment, the CDSS disclosed herein can be in the form of a software application or program that represents a data processing method.

In one embodiment, the CDSS includes a rule-based framework in the form of a directed graph. The directed graph can include one or more node/vertex as the fundamental unit and an edge as the connector between nodes and pointing in a specific direction, for example A>B which means A can travel to B but not vice versa. As such, the CDSS and rule-based or knowledge-based approach can be in the form of graph rules that are coded to the software. These rules direct what question appears next to the user based on the answer of previous question, the rules that generate the diagnosis and treatment plan options.

In one embodiment, the CDSS can include a rules engine with a rule-based framework in the form of a directed acyclic graph. The rule-based framework is a JavaScript frontend framework where the rules can be stored in an XML file or a graph database. The JavaScript frontend will parse the rules on load and subsequently depending on the user's selection in the JavaScript frontend, the JavaScript will help to traverse the graphs and return the outputs based on the logic indicated in the rules.

In one embodiment, the CDSS disclosed herein can be built upon and developed through the use of a graph data structure simulating the knowledge-based data within the domain.

The rule-based or knowledge-based framework of the CDSS can be derived from information found in industry accepted medical literature, such as textbooks, journals etc. Accordingly, this knowledge-based approach can incorporate academic research and studies, clinical expert knowledge and industrial best practices that for any given mouth condition there are ethical steps to provide treatments to the patient. The clinical expert knowledge can be derived from renowned professors, and expert specialists.

The knowledge-based approach of the CDSS focuses on creating a knowledge description language which, when combined with a reasoner, can make diagnostic inferences.

The CDSS disclosed herein can be based on a comprehensive step-by-step protocol or rule-based graph to assist users (e.g. clinical professionals) to intuitively and systematically collect and analyse personal and clinical data of patients to develop comprehensive and individualised care and treatment plans. The advantage of using the CDSS over machine learning tools (e.g. supervised learning) is it's clinical interpretability where each decision step is clearly identified and traceable, unlike other “black box” algorithms.

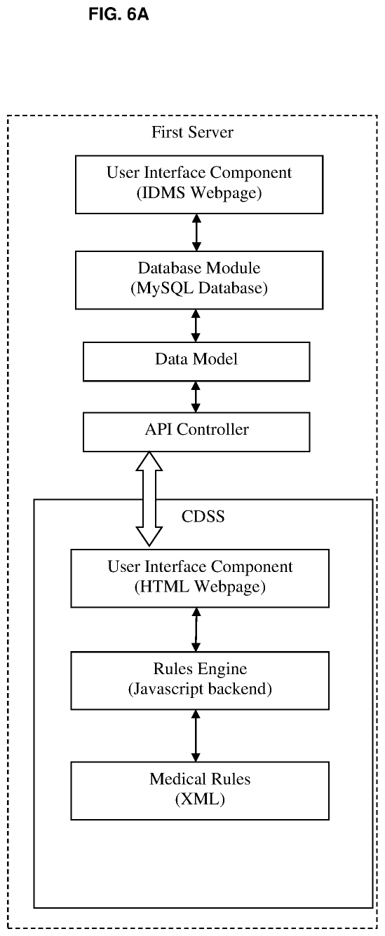

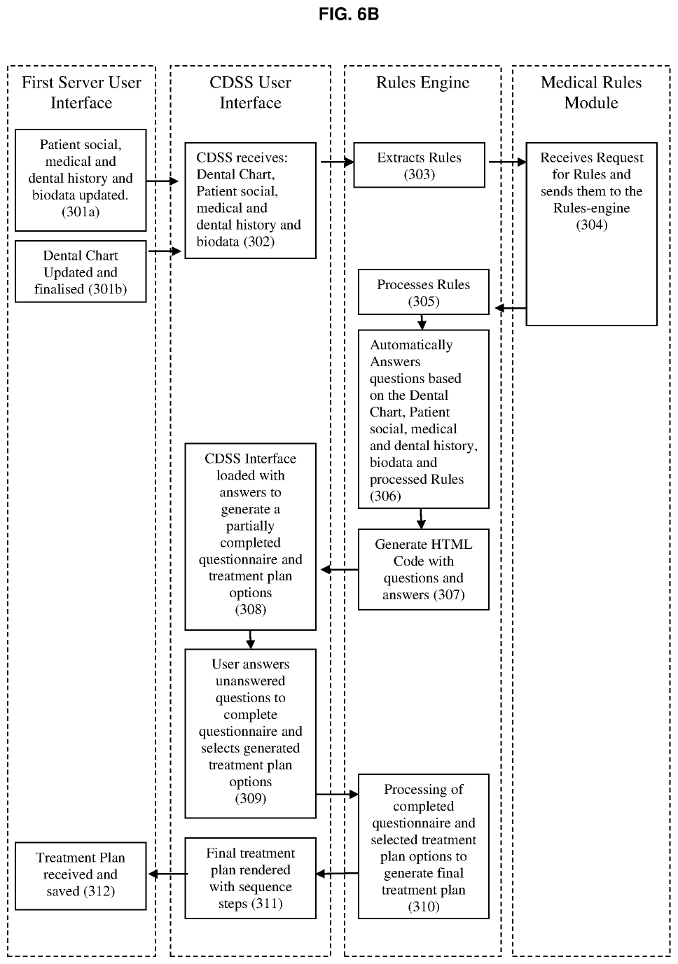

FIG. 6A is a representative embodiment of the CDSS disclosed herein in relation to the first server. The first server can include a user interface component for a user to view patient social, medical and dental history, biodata as well as the dental chart, and update the same if necessary. The first server user interface component can include an IDMS webpage. The patient's social, medical and dental history, biodata as well as the dental chart can be managed and extracted by a database module. The database module can be a MySQL database module. A data model can be included in the first server to organize the data received from the database module and standardise how they relate to one another.

The CDSS can include a CDSS user interface component for a user to view the generated questionnaires, treatment plan options and final treatment plan, and complete or select information therein. The CDSS user interface component can include a HTML webpage. A rules-engine can be employed that uses rules in a JavaScript framework. Further, a medical rules module can be included that can define the rules in an Extensible Markup Language (XML).

An API controller can be included for handling requests between the first server and CDSS, more specifically from the CDSS user interface and the data model in the first server, where the data model simply manages the data structure based on the logic specified.

FIG. 6B flowchart and operational relationship of the CDSS between computational components as well as the input from the user and how they interact with the CDSS.

At step 301a, the patient's social, medical and dental history and biodata can be viewed and updated by the user via the first server user interface. Similarly, at step 301b the populated dental chart can be updated and finalised by the user via the first server user interface. The user can subsequently request for the CDSS to process the input from steps 301a and 301b.

At step 302, the CDSS user interface receives the input from steps 301a and 301b. At step 303, the CDSS rules-engine requests that the medical rules be extracted from the medical rules module for processing. At step 304, the medical rules module receives the request from the rules engine and sends the rules to the rules-engine, at step 305.

At step 306, the rules engine automatically answers questions using the information received at steps 301a and 301b based upon the rules processed. The rules engine generates a HTML code with questions and answers to be sent to the CDSS user interface at step 307.

At step 308, the CDSS user interface is loaded with the answers from step 307 to generate a partially complete questionnaire and treatment plan options.

At step 309, the user can check the answered questions and complete the unanswered questions to submit a completed dental questionnaire. Once the questionnaire has been completed the treatment plan options are generated for the user selection.

At step 310, the rules engine processes the completed questionnaire and selected treatment plan options to generate the final treatment plan with sequence of steps. The final treatment plan is rendered and sent to the CDSS user interface for the user to review at step 311. The final treatment plan is received and saved by the first server user interface.

### User Interface (Dashboard)

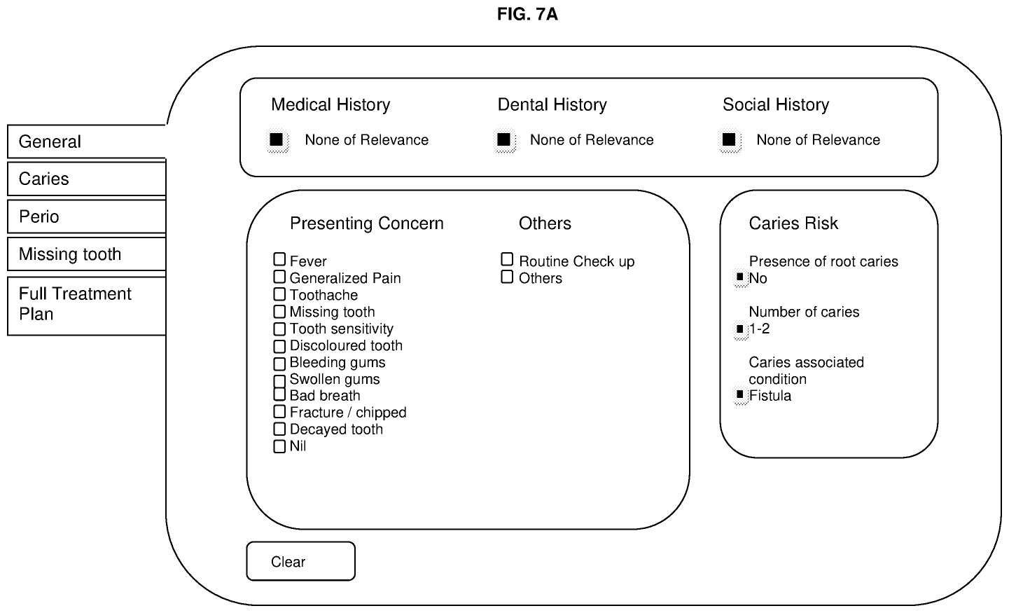

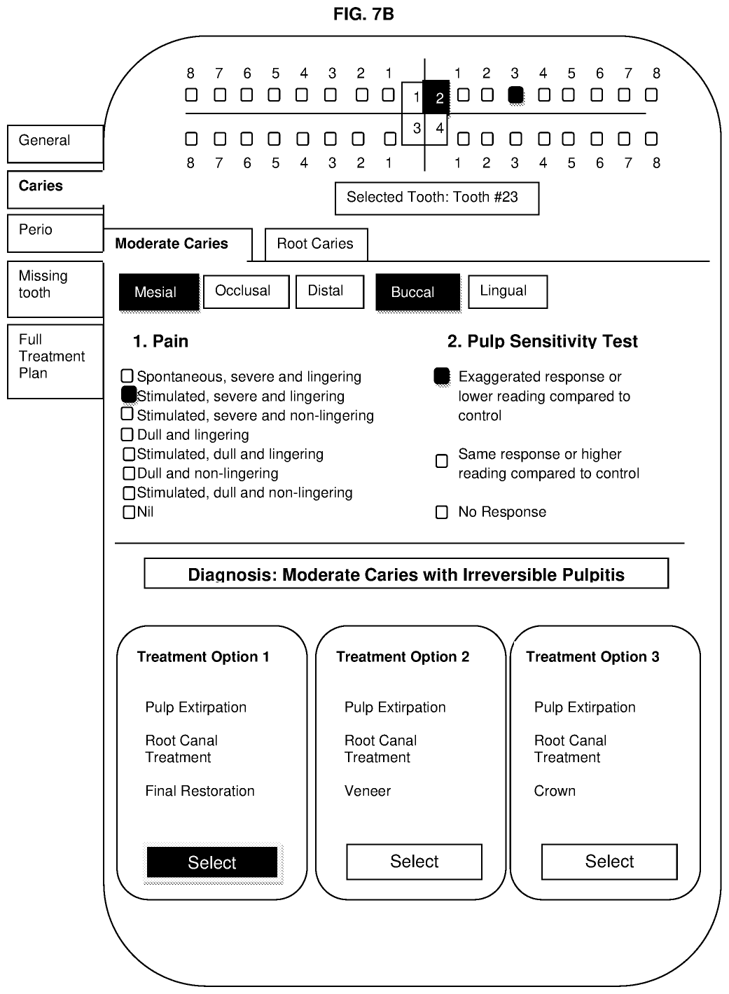

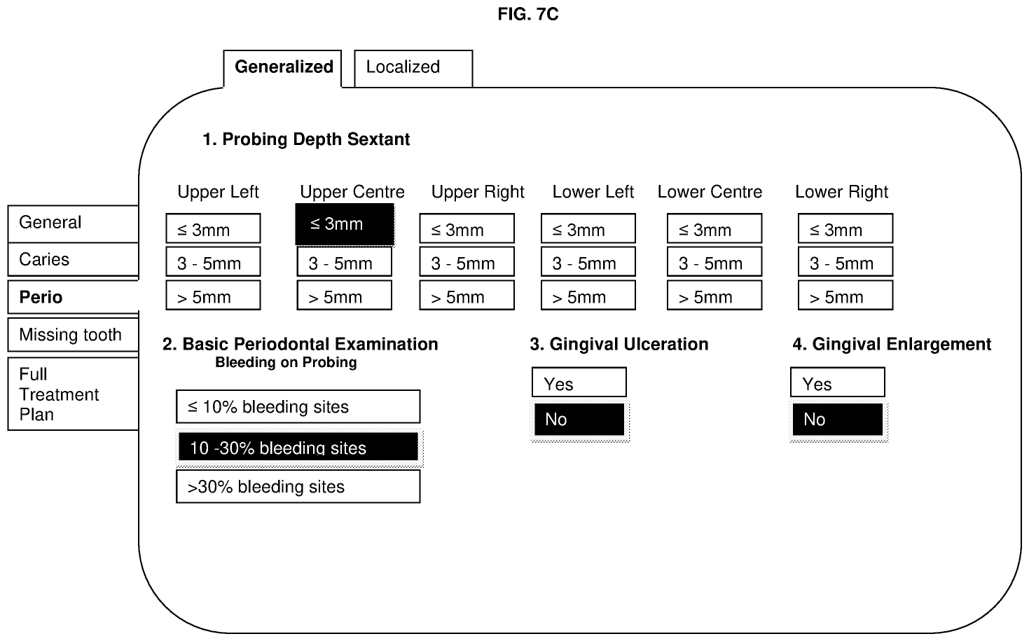

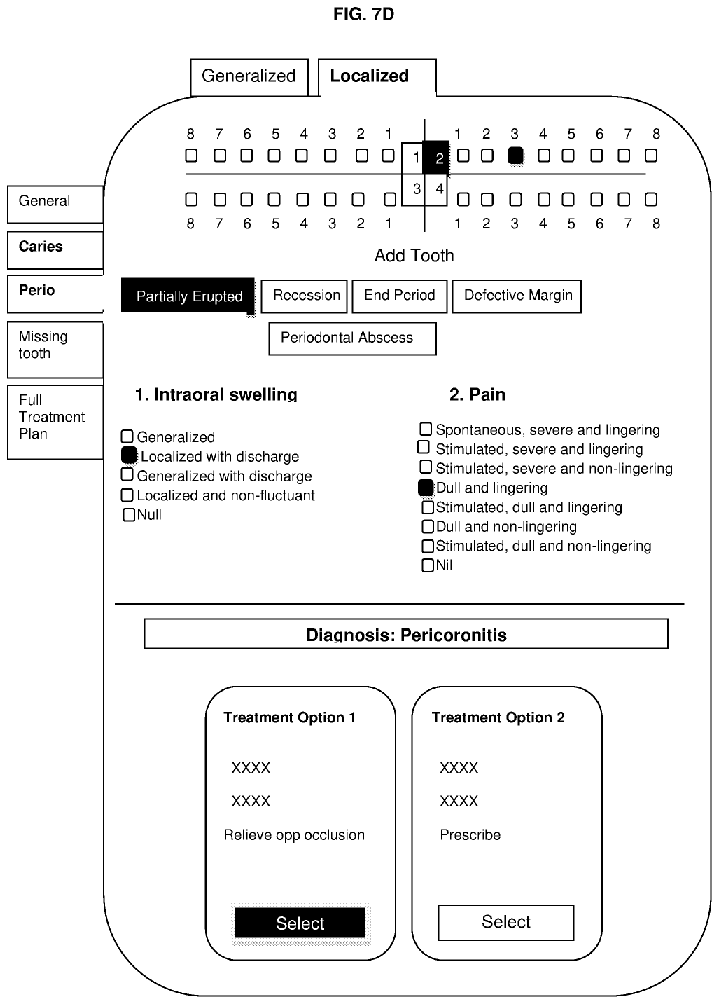

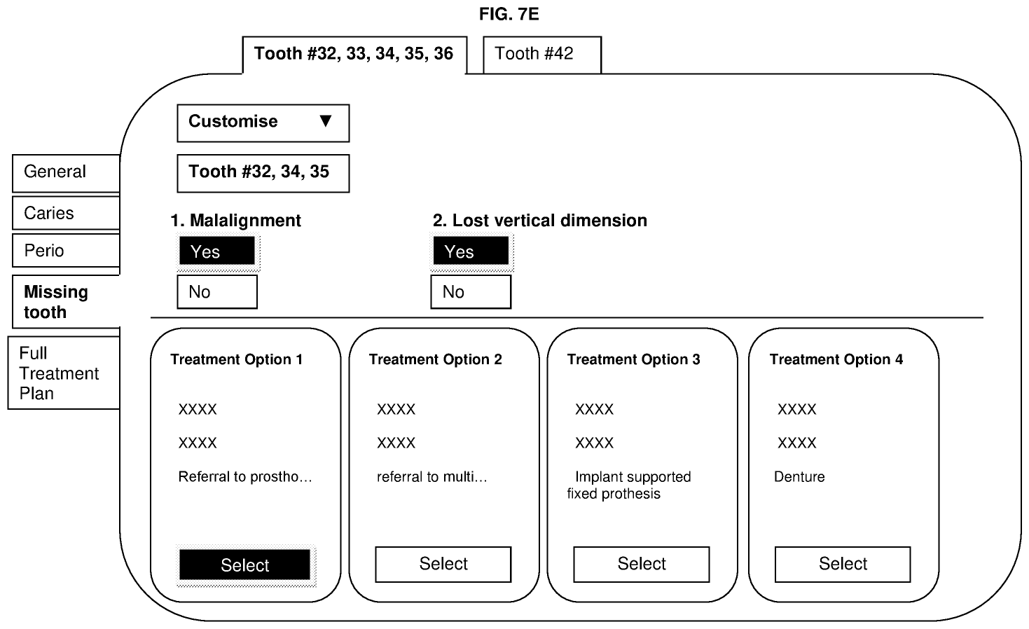

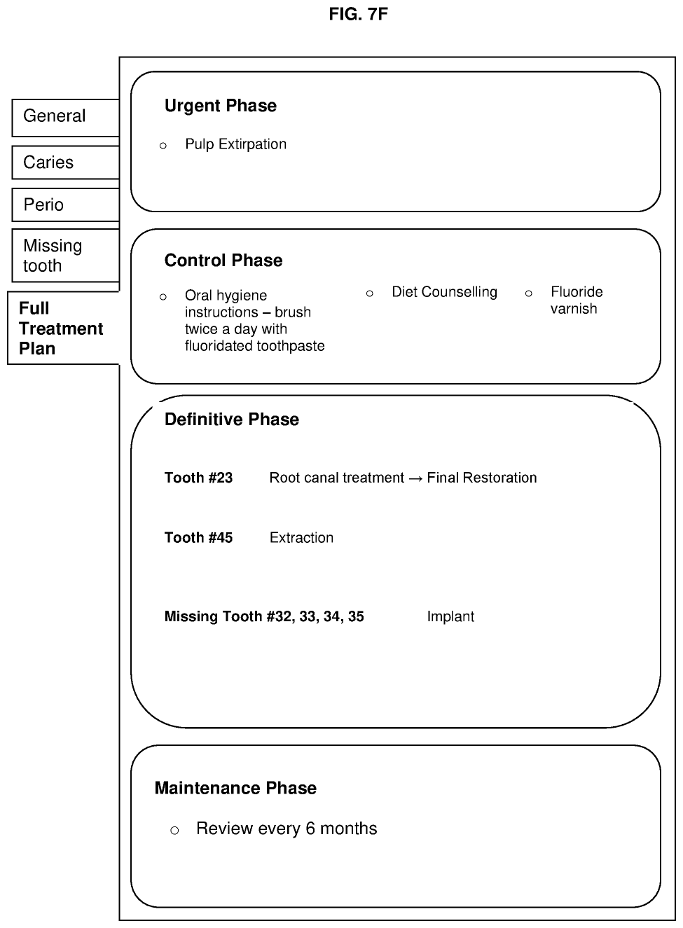

As shown in FIG. 7A-F, the information and output from the AI model and CDSS can be displayed to the user via the first server and a user interface or dashboard. As indicated in FIGS. 6A and B, this user interface can include a first server user interface and a CDSS user interface.

In particular, the user interface/dashboard can present information through separate categories relating to patient data, dental issues or conditions and treatment plan. This dashboard can be available to the user in the form of a web accessible page identified by its URL, or as an application running on a computer. The dashboard can be operational to present the partially completed questionnaire and allow the user to input and complete the questions.

As shown in FIG. 7A, a functional tab labelled “General” can relate to a patient information display area including options to select a number of symptoms and risks of the patient as well as the relevance of the patient biodata on their medical, social or dental history.

FIG. 7B-E show separate tabs with dedicated questions directed to dental issues identified by the AI model and indicated on the final dental chart. Each tab can be provided for each dental issue identified with a pre-set selection and series of questions related to the symptoms of the dental issue to be completed and reviewed by the user. Following completion of the questions related to the symptoms by the user, a diagnosis is provided along with one or more treatment options for the user to select one. For example, in FIG. 7B a tab for detected caries is shown with a representative dental chart indicating the caries relates to tooth number 23, the caries can be differentiated as either moderate or root caries followed by the location of said caries (mesial, occlusal, distal, buccal, lingual), the user can then select symptoms (pain) of the caries in numbered questions 1 and 2 that provide the diagnosis and three treatment options to choose from. In FIGS. 7C and 7D, a tab for detected periodontal disease is shown, whereby this periodontal disease can be differentiated into generalised (whole mouth) or localized (per tooth), each of which includes one or more questions related to the location (left, right, upper, lower), size (<3 mm, >5 mm) and symptoms (ulceration, swelling, pain) of said periodontal disease to answer in order to provide a diagnosis and two treatment options to choose from. In FIG. 7E a tab for one or more detected missing tooth is shown, whereby questions are provided on the symptom (alignment, dimension) of said missing tooth in order to provide a diagnosis and four treatment options to choose from.

Once a treatment option has been selected for each dental issue and all necessary questions answered, the completed questionnaire can be further processed by the CDSS to generate a final (Full) treatment plan with a sequence of treatment steps. The sequence of treatment steps can be in the form of one or more phases including but are not limited to an “urgent phase”, “control phase”, re-evaluation phase”, “definitive phase” and a “maintenance phase”. The “urgent phase” relates to the need for an emergency treatment of an identified dental issue, however, as will be appreciated the treatment plan may not include an “urgent phase” if no emergency treatment is required.

In FIG. 7F, an exemplified final treatment plan is shown with the sequence of treatment steps. As will be appreciated, the phases included in the final (full) treatment plan are dependent upon the answered questions. For example, in FIG. 7B, if the user selects option 2 instead of option 1, a re-evaluation phase will appear in the final treatment plan.

Accordingly, in one embodiment the sequence of treatment steps can be 1. Urgent phase; 2. Control phase; 3. Re-Evaluation phase; 4. Definitive phase; and 5. Maintenance phase. In another embodiment, the sequence of treatment steps can be 1. Control phase; 2. Re-Evaluation phase; 3. Definitive phase; and 4. Maintenance phase. In another embodiment, the sequence of treatment steps can be 1. Urgent phase; 2. Control phase; 3. Definitive phase; and 4. Maintenance phase.

The Urgent or Emergency Phase (UP) can relate to patients who present with fever, swelling, pain, bleeding, or infection needs to be treated with urgency.

The Control Phase (CP) can be planned to a) eliminate active disease such as caries and inflammation; b) remove conditions preventing maintenance; c) eliminate potential causes of disease, and d) begin preventive dentistry activities such as i) Management of Gingival and Periodontal Infection; ii) Management of Caries Risk; and iii) Management of teeth with caries.

The Re-Evaluation Phase (RP) can refer to a holding stage that allows for resolution of inflammation and time for healing. Accordingly, during this phase home care habits are reinforced, motivation for further treatment is assessed, and initial treatment and pulpal responses are re-evaluated before definitive care can begin.

The Definitive Phase (DP) can refer to a stage for correcting and resolving certain dental issues. This phase may need periodontal surgery, oral surgery, replacement of missing teeth. In one embodiment, the management of missing Tooth/Teeth is in this definitive phase.

The Maintenance Phase (MP) can include a regular recall time frame for review and subsequent examinations that: a) may reveal the need for adjustments to prevent future breakdown, and b) provide an opportunity to reinforce home care. This phase can indicate that a review of the patient should be carried out every 3, 6 or 12 months dependent on the stage of development and severity of the identified dental issue. For example, i) a review every 3 months will be stated when the patient has Stage 3 and Stage 4 Periodontitis or High Caries Risk; ii) a review every 6 months will be stated when the patient has Medium Caries Risk and Stage 1 or 2 Periodontitis; iii) a review every 12 months will be stated when the patient is a Low Caries Risk or in Periodontal Health.

Depending on the treatment recommendation, the user can make decisions based on the CDSS and its recommendation in conjunction with their own judgment and experience. In addition, the output results of the CDSS can be checked and verified for consistency with the decision map using visual checks and standard testing (e.g. unit testing, integration testing) to ensure robustness. Further, the CDSS has been validated by a group of specialists to test the comprehensiveness and accuracy of the diagnosis and recommended treatment plan generated.

### WORKING EXAMPLES

The following non-limiting examples are provided for illustrative purposes only in order to facilitate a more complete understanding of representative embodiments now contemplated. These examples are intended to be a mere subset of all possible contexts in which the components of the system and steps of the method disclosed herein may be combined. Thus, these examples should not be construed to limit any of the embodiments described in the present specification, including those pertaining to the type and amounts of components of the system and/or methods and uses thereof.

In a dental clinic setting, a patient may visit the clinic with one or more dental problems. The dentist will request for an X-ray (orthopantomogram) to better understand the patient's dental condition.

This X-ray and other patient data can be fed into the computer system disclosed herein. In this regard, the system can be embedded into a Patient Management System (PMS) of the clinic. The system will provide an output within seconds that indicates the mouth problems that the patient has. Often when the patient comes in with a pain, the dentist's immediate priority is to look at a singular pain area in the X-ray and overlook other dental problems.

By feeding the X-ray image through the AI models of the system disclosed herein, a holistic analysis is performed to potentially detect multiple mouth problems within seconds. As a consequence, a dental chart is automatically populated with AI predictions of mouth conditions that the dentist is able to immediately review with the patient. For example, the patient may come to the clinic with a toothache, but upon analysis by the system, an additional 4-5 interrelated problems may be detected.

The dental problems detected combined with further clinical examination by the dentist, will be further processed by the CDSS disclosed herein. The CDSS will generate a questionnaire for the dentist to answer with treatment plan options for selection by the dentists. Following the selection of treatment options and completion of the questionnaire, a final treatment plan with a proper sequence will be generated for all detected dental issues.

The correct sequence of treatment steps is important because sometimes the success rate of the treatment is determined by a correct sequence. For example, an implant will have a higher success rate if the gum/bone problem has been resolved first. If an implant is placed in a mouth with a poor oral hygiene condition including some gum/bone problem, the possibility of implant failure will increase. Thus, the system and method disclosed herein will generate the correct sequence relating to treating the gum followed by proceeding with the implant placement.

The analysis of patient data (X-ray images) using AI models and the generation of a treatment plan using a CDSS will be completed during the initial visit of the patient. That means the patient visits the dental clinic for a potential mouth condition or disease and will be provided with a complete holistic treatment plan to resolve the patient dental condition. Further, any serious dental issues will be detected early or prevented through a holistic treatment plan.

Accordingly, the system and methods disclosed herein aims to improve the standard of dentistry practice across the industry. Patients will no longer receive information and advice solely on their primary mouth complaint, but they will receive a comprehensive and holistic, yet personalized diagnosis and treatment planning for their mouth condition. Patients can be assured of more accurate diagnosis and the best treatment options while avoiding possible errors based on personal judgement and bias. Furthermore, early detection of dental diseases can be achieved. This will help patients to manage their cost of treatment and at the same time, it increases the awareness and the knowledge of good dental healthcare regime in the community.

### Example 1

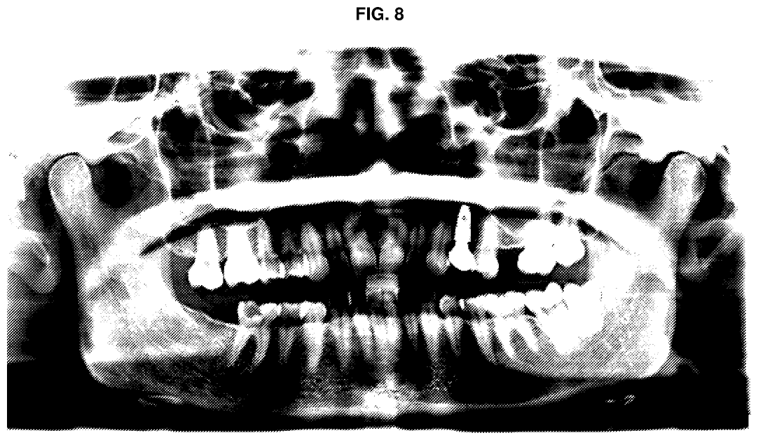

FIG. 8 shows an exemplified X-Ray image that was input to the system and method disclosed herein to analyse the patients mouth condition and generate a treatment plan in accordance to the representative method steps outlined in FIG. 3.

Initially, during examination of a patient the dentist took a full mouth X-Ray. The X-Ray was uploaded to the system and displayed to the dentist via a user interface for review. The system subsequently analysed the X-Ray image upon request by the dentist by employing the AI model.

The result of the AI model analysis was used to populate a dental chart listing the tooth number with an indicated dental issue along with additional pertinent information and details. The dental chart was displayed to the dentist via a user interface, whereby the dentist updated and made any changes to said dental chart upon further examination of the patient.

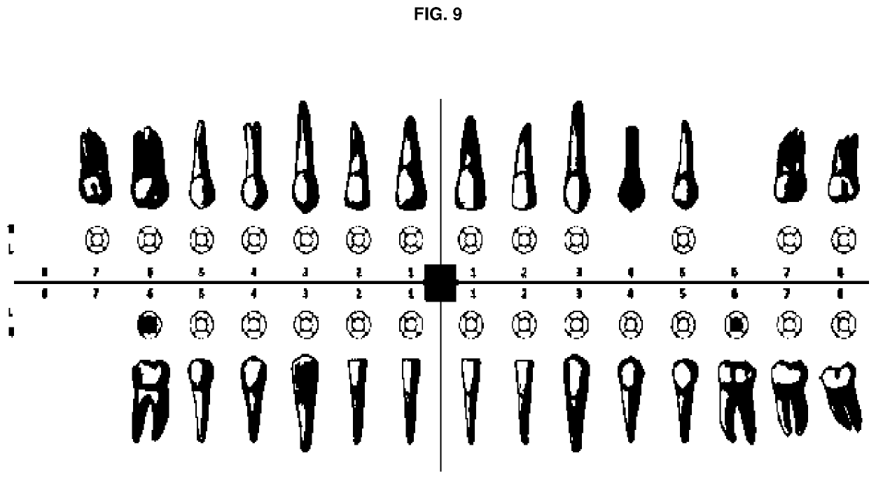

The final dental chart populated is shown in FIG. 9 and was accompanied by a table populated by the AI predictions, as shown in Table 1 below.

TABLE 1

| Tooth Number | Details | Symptoms |
|---|---|---|
| 18 | Missing | — |
| 24 | Implant | — |
| 24 | Crown | Crown |
| 26 | Missing | — |
| 36 | Filling | Filling - O |
| 38 | Eruption | Impacted |
| 46 | Caries | Extensive Caries - D |
| 46 | Filling | Filling with Caries - O |
| 47 | Missing | — |
| 48 | Missing | — |

Based on the final dental chart, a partially complete dental questionnaire was subsequently generated that required the dentist to answer questions in relation to each tooth and symptoms. The questionnaire was displayed to the dentist via a user interface that can allow the dentists to complete the questionnaire.

The dental questionnaire included the following multiple choice questions for selection, as shown in Table 2 below specifically in relation to an issue with tooth #46. As will be appreciated, the questionnaire may vary for the other teeth identified with issues dependent upon that issue and relevant details.

TABLE 2
Tooth # 46

Q1. Pain
Spontaneous, severe and lingering
Stimulated, severe and lingering
Stimulated, severe and non-lingering
Dull and lingering
Stimulated, dull and lingering
Dull and non-lingering
Stimulated, dull and non-lingering
Nil
Q2. Radiolucency In Crown
In enamel
In outer third of dentin
In middle third of dentin
Extending to inner third of dentin
Approximating or in pulp
None of relevance
Q3. Pulp Sensibility Test
Exaggerated response or lower reading compared to
control
Same response or higher reading compared to control
No response

Following completion and submission of the questionnaire, a diagnostic assessment and one or more treatment plan options were generated by the CDSS and displayed to the dentist via a user interface. Table 3 below outline the information provided to the user in relation to tooth #46 and missing teeth 26 and 47.

TABLE 3
Tooth 46 Diagnosis - Extensive Caries with irreversible pulpitis

| | | |
|---|---|---|
| Treatment Option 1 | Treatment Option 2 | Treatment Option 3 |
| (a) tooth 46-pulp extirpation | (a) tooth 46-pulp extirpation | (a) Tooth 46-Extraction |
| (b) tooth 46-Root Canal | (b) tooth 46-Root Canal | |
| treatment | treatment | |
| (c) Tooth 46-Review 2-4 weeks | (c) Tooth 46-Review 2-4 weeks | |
| after completion of root filing | after completion of root filing | |
| (d) Tooth 46-Onlay restoration | (d) Tooth 46-Crown restoration | |
| Comment: | Comment: | Comment: |
| When the pulp is inflamed to an | When the pulp is inflamed to an | Replacement of the extracted |
| extent that it is incapable of | extent that it is incapable of | tooth can be considered |
| healing (irreversible pulpitis) or | healing (irreversible pulpitis) or | |
| where the tissues of the pulp are | where the tissues of the pulp are | |
| dead (necrosis) or when the | dead (necrosis) or when the | |
| toxins have reached the bony | toxins have reached the bony | |
| tissue beyond the root tip to | tissue beyond the root tip to | |
| cause inflammation and bone | cause inflammation and bone | |
| destructions (apical periodontitis), | destructions (apical | |
| root canal treatment is needed | periodontitis), root canal | |
| to clean the system off the | treatment is needed to clean the | |
| infectives tissues and seal with a | system off the infectives tissues | |
| root filing. When the loss of tooth | and seal with a root filing. When | |
| tissues in the crown due to caries | the loss of tooth tissues in the | |
| has caused the cusp/s to be | crown due to caries is extensive, | |
| weakened, an only/restoration is | a crown restoration is needed to | |
| needed to protect the cusp/s | restore back shape, size and | |
| from fracture under function. | function. | |
| Plans: | Plans: | Plans: |
| Tooth 46 - pulp extirpation | Tooth 46 - pulp extirpation | Tooth 46 - Extraction |
| Tooth 46 - Root Canal treatment | Tooth 46 - Root Canal treatment | |
| Tooth 46 - Review 2-4 weeks | Tooth 46 - Review 2-4 weeks | |
| after completion of root filing | after completion of root filing | |
| Tooth 46 - Onlay restoration | Tooth 46 - Crown Restoration | |

Missing Tooth Group 26
Implant-supported single crown
Fixed-fixed bridge
Referral to an orthodontist for orthodontic space closure
Removable partial denture
Missing Tooth Group 47
Implant-supported single crown
Removable partial denture

Following selection by the dentist of the treatment plan option for each tooth and identified dental issue, the CDSS generated a final treatment plan with a sequence of treatment steps taking into consideration the identified problems with all teeth for a holistic treatment plan and sequence of steps. The treatment steps were divided into phases and comprise an urgent phase, control phase, re-evaluation phase, definitive phase and maintenance phase, as shown in Table 4 below.

TABLE 4

Patient Information
Name:
Age: >20
Urgent Phase
Tooth 46 - Pulp extirpation
Control Phase
Scaling & Polishing
Oral Hygiene Instructions - Brush twice daily with the
fluoridated toothpaste (>1,450 ppm)
Diet Counselling
Apply fluoride varnish
Tooth 46 - Root Canal Treatment
Re-Evaluation Phase
Tooth 46 - Review 2-4 weeks after completion of the root
filling
Definitive Phase
Tooth 46 - Onlay Restoration
Replacement of Missing Tooth 26
(a) implant-supported single crown
Replacement of missing tooth 47
(a) implant-supported single crown
Maintenance Phase
Review patient every 6 months

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The foregoing has described the principles, embodiments and. modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

The invention has been described broadly and generically herein. Each of the narrower species and subgeneric groupings falling within the generic disclosure also form part of the invention. This includes the generic description of the invention with a proviso or negative limitation.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

What is claimed is:

1. A computer-implemented method for generating a dental treatment plan of a patient comprising the steps of:
receiving patient data comprising a dental image of the patient;
analysing the dental image of the patient using an AI model to generate AI predictions on tooth detection, numbering and dental issues of the patient;
populating a dental chart based on the AI predictions and input received from one or more users;
generating a completed dental questionnaire using a clinical decision support system (CDSS) based upon the populated dental chart, patient data and input received from the one or more users;
generating a final treatment plan based on the completed dental questionnaire; and
displaying the final treatment plan to the one or more users.

2. The computer-implemented method of claim 1, wherein the AI prediction is based upon the following components:
bounding box x-min, y-min coordinates with a width and height;
a probability of an object being present in the bounding box; and
class probabilities of the object detected in the bounding box.

3. The computer-implemented method of claim 1, wherein the AI model comprises a first deep learning CNN, for detecting objects that are dynamic in size and a second deep learning CNN for identifying objects that are static in size.

4. The computer-implemented method of claim 3, wherein the first deep learning CNN comprise a CenterNet model for detecting objects that are dynamic in size the second deep learning CNN comprise an EfficientDet model for identifying objects that are static in size.

5. The computer-implemented method of claim 1, wherein the CDSS is a rule-based or knowledge-based CDSS.

6. The computer-implemented method of claim 5, wherein the rule-based CDSS comprises a framework in the form of a directed graph.

7. The system of claim 18, wherein the knowledge-based CDSS comprises a framework in the form of a graph rule coded to the software, focusing on creating a knowledge description language, for making diagnostic inferences.

8. A computer system comprising a processor configured to perform the method of claims 1 to 7.

9. A computer program comprising instructions which, when the program is executed by a computer, cause the computer to carry out the method of claims 1 to 7.

10. A computer-readable storage medium comprising instructions which, when the program is executed by a computer, cause the computer to carry out the method of claims 1 to 7.

11. A system for generating a dental treatment plan of a patient comprising:
an input unit for receiving patient data comprising a dental image;
a computer-readable storage medium configured to store instructions defining an AI model;
a first server to execute a clinical decision support system (CDSS);
a second server to execute the instructions defining the AI model,
wherein the second server is configured to perform operations comprising:
generate AI predictions on tooth detection, numbering and dental issues of the patient, based upon the dental image using the AI model; and
populate a dental chart based on the AI predictions and input received from one or more users,
wherein the first server is configured to perform operations comprising:
generate a completed dental questionnaire based upon the dental chart and input received from one or more users using the CDSS; and
generate a final treatment plan based the completed dental questionnaire using the CDSS; and
an output unit configured to communicate the dental chart, questionnaire and final treatment plan to the user.

12. The system of claim 1, wherein the AI model combines image recognition and localisation for object detection of an input dental image to detect and identify teeth locality for numbering and classification of the teeth relating to dental issues.

13. The system of claim 12, wherein the locality of teeth and tooth numbering is determined by forming of bounding boxes around teeth.

14. The system of claim 13, wherein the AI predictions is based upon the following components:
bounding box x-min, y-min coordinates and the width and height;
the probability of an object being present in the bounding box; and
the class probabilities of the object detected in the bounding box.

15. The system of claim 14, wherein the bounding boxes defines a boundary for each tooth captured in the dental image and assigns a number to each detected tooth.

16. The system of claim 11, wherein the AI model comprises a first deep learning CNN, for detecting objects that are dynamic in size and a second deep learning CNN for identifying objects that are static in size.

17. The system of claim 16, wherein the first deep learning CNN comprise a CenterNet model for detecting objects that are dynamic in size the second deep learning CNN comprise an EfficientDet model for identifying objects that are static in size.

18. The system of claim 11, wherein the CDSS is a rule-based or knowledge-based CDSS.

19. The system of claim 18, wherein the rule-based CDSS comprises a framework in the form of a directed graph.

20. The system of claim 18, wherein the knowledge-based CDSS comprises a framework in the form of a graph rule coded to the software, focusing on creating a knowledge description language, for making diagnostic inferences.
